(19) United States
(12) Patent Application Publication
(10) Pub. No.: US 2005/0239195 A1
(43) Pub. Date: Oct. 27, 2005

(54) Reaction chamber

(75) Inventors: Guy R J Oram, Kingston (CA); Andreas Brunner, Oberembrach (CH); Rupert Hagg, Bassensdorf (CH)

Correspondence Address: LADAS & PARRY, 26 WEST 61ST STREET, NEW YORK, NY, 10023, US

(73) Assignee: MILLENIUM BIOLOGIX AG, CH-8952, SCHLIEREN (CH)

(21) Appl. No.: 10/517,496

(22) PCT Filed: Jun. 10, 2003
(86) PCT No.: PCT/IB03/02518

§ 371 Date: Jun. 24, 2005

Related U.S. Application Data
(60) Provisional application No. 60/388,482, filed on Jun. 13, 2002.

(51) Int. Cl. C12M 1/34

(52) U.S. Cl. 435/288.3; 435/293.1; 435/287.2

(57) ABSTRACT

A reaction chamber assembly comprising a e.g. microscope slide or any other slide or carrier system and an assembly cover is described, wherein said assembly cover comprises at least one port and at least one channel having a first end at the port and a second end at a reaction compartment which reaction compartment together with the e.g. microscope slide forms a reaction chamber with predetermined volume.

### CROSS REFERENCES TO RELATED APPLICATIONS

This application claims the priority of provisional patent application 60/388 482, filed Jun. 13, 2002, the disclosure of which is incorporated herein by reference in its entirety.

### FIELD OF THE INVENTION

The present invention relates to the development of a reaction chamber for temperature controlled reactions of biological specimens in a defined volume and at defined temperatures as necessary for hybridization reactions with nucleic acids or detection of proteins or antibodies.

The present invention furthermore relates to a reaction chamber or even a small-scale bioreactor system enclosing a pre-defined volume, herein a microscope slide carrying the biological specimen and an assembly cover act as the essential parts. The integration of heating devices, the adjustment to fluid pathways and the possibility of computer control make the system suitable for high throughput applications.

### BACKGROUND OF THE INVENTION

The microarray technology or DNA-chip technology which allows expression monitoring of hundreds or thousands of genes simultaneously have become an established and powerful molecular biology tool during the last couple of years. Using this technology, hybridization of a polynucleotide probe on the array and a complementary polynucleotide from the sample to form a stable duplex through base pairing is an essential step. These target molecules are labeled either with fluorescence dyes or with radioactive isotopes, whereby the latter requires a safe incubation system.

While in the past hybridization experiments were performed primarily using nylon or nitrocellulose membranes for dot blot applications, northern and southern hybridization experiments, the polynucleotides of microarrays are mainly spotted onto precoated glass or plastic substrates.

Such rigid microarrays as well as nylon or nitrocellulose microarrays contain a matrix (array) of either spotted single stranded oligomeric DNA or cDNA spots representative of a particular gene. Associated with the above mentioned technologies are systems that allow hybridization of the probe with target sequences to investigate RNA probe molecules of specific tissues or cells.

Since the microarray technology is moving nowadays towards the use of these rigid microarrays made either of glass or plastic, the process of hybridization has also changed substantially. In principal, due to the material properties of rigid microarrays it has become possible to dramatically downsize the hybridization volume in order to conserve valuable sample material. Other important features are safety, simplicity and cost-effectiveness. According to these criteria, all existing technologies exhibit one or more weaknesses.

Further, a recently launched product by Clontech, a chip based antibody array has paced the way for similar applications in the field of proteomics. As in the hybridization procedure, similar experimental steps like blocking, specific protein protein reaction, washing, and detection have to be addressed. Currently, there is no system on the market that can be considered as an “all-in-one reaction system” for these various applications that is easy to handle, affordable in price, allows temperature control and can be used also for radioactivity.

### DISCLOSURE OF THE INVENTION

Thus, one object of the present invention is to provide a reaction chamber assembly comprising e.g. a microscope slide or any other slide or carrier system and an assembly cover, wherein said assembly cover comprises at least one port and at least one channel having a first end at the port and a second end at a reaction compartment which reaction compartment together with the microscope slide forms a reaction chamber with predetermined volume.

A further object of the present invention is to provide a modular system comprising at least two reaction chamber assemblies of the invention, wherein any one of the bioreactors can individually be removed/replaced.

Still a further object is to provide an assembly cover comprising at least one port and at least one channel having a first end at the port and a second end at a reaction compartment which reaction compartment together with the microscope slide forms a reaction chamber with predetermined volume.

Still a further object is to provide a temperature controlling and adjusting cover with an at least in part planar surface that can be brought in contact with the microscope slide or the assembly cover of the bioreactor of the present invention in at least the region of the reaction chamber.

Yet another object is a temperature controlling and adjusting system comprising at least two temperature controlling and adjusting covers.

Hybridization of microarrays in general involves a system where a low amount of volume sample is incubated in the presence of the target sequences at defined temperatures. The reaction chamber assembly is suited not only for hybridization procedures of nucleic acid material mounted on glass slides but can also be used for all kind of protein binding assays, e.g. immunological assays. A recently launched product by Clontech—a chip based antibody array—may become a breakthrough technology in the field of proteomics.

The reaction chamber of this invention allows to perform the various incubation procedures (e.g. protein-antibody reaction, hybridization, washing steps, detection) within an all-in-one system, making such reactions more easily, more accurate and reducing the consumption of valuable sample material.

The reaction chamber described in the current invention is easy-to-use and therefore ideally suited for working with radioactively labelled material.

Due to its modular construction system it qualifies well for adaptation to automatic pipette devices and thus for higher throughput applications and it can be designed to be disposable. The reaction chamber assembly described in the present invention can be designed to include a heating such as a conductive wire and/or a thermoelement connectable or connected to a heat control system for exact and individual temperature control. This allows to perform a temperature controlled reaction as a stand-alone system which is not described in the inventions U.S. Pat. No. 6,159,727, U.S. Pat. No. 5,346,672.

If internal temperature control is not needed, a further important feature comes into operation: Since the cover assembly is preferably made of a thermoconductive material, simple and cheap temperature control can be obtained by placing the bioreactor into usual lab devices such as thermocyclers or hybridization ovens.

### BRIEF DESCRIPTION OF THE FIGURES

The present invention will be further understood from the following description with reference to the figures, in which

FIG. 1: is a perspective view of a reaction chamber assembly in accordance with the invention.

FIG. 2: is a longitudinal section through a reaction chamber assembly similar to the perspective view of the assembly shown in FIG. 1.

FIG. 3: is a front view of the perspective view of a reaction chamber assembly similar to the ones shown in FIGS. 1 and 2.

FIG. 4: shows a fluid flow schematic for automated use of the reaction chamber assembly of the invention.

FIG. 5: illustrates possible mechanisms of integrated heating devices.

FIG. 6: shows the assembly of mulitple reaction chamber assemblies as stand alone modules where a heat control processor is used for individual temperature regulation.

FIG. 7: shows a schematic drawing of an example for a high through put hybridization system where multi sample loading is combined with modular reaction chambers and different fluid pathways. Preincubation-, blocking-, washing reactions, temperatur control and sample loading from a multiwell plate can be performed for each sample individually.

FIG. 8: shows a specific embodiment of a channel end.

FIG. 9: shows a temperature profile measured with an NTC thermistor in a modified reaction chamber. The temperature profile on the left hand side illustrates a temperature over time diagram for a target temperature of 42° C., whereas the other diagram shows a similar temperature curve for 50° C.

FIG. 10: A reaction chamber is shown in a TGradient PCR thermal cycler (Whatman Biometra GmbH, Göttingen, Germany) prior to hybridization.

FIG. 11: This is a typical result of a microarray hybridized in a reaction chamber of the present invention. The microarray was scanned in an Affymtrix 418 microarray scanner.

### MODES FOR CARRYING OUT THE INVENTION

The present invention comprises a reaction chamber assembly 1 usable as small-scale bioreactor with a reaction chamber 2 enclosing a pre-defined volume, wherein e.g. a microscope slide or any other slide or carrier system 3 optionally carrying biological specimen, e.g. proteins, nucleic acids or cells, and an assembly cover 4 act as the essential parts. Pressing the microscope slide 3 onto the assembly cover 4 results in a ready-to-use system that allows to perform any kind of biological reactions, preferably those requiring a predefined volume and temperature control.

A reaction chamber assembly 1 at least comprises a microscope slide 3 and an assembly cover 4, wherein said assembly cover 4 comprises at least one port 5 and at least one channel 6 having a first end at the port 5 and a second end 8 at a reaction compartment 9 which reaction compartment 9 together with the microscope no slide 3 forms a reaction chamber 2 with predetermined volume.

In preferred embodiments, the assembly cover 4 comprises an O-ring 10 surrounding the reaction compartment 9 and establishing a seal to the microscope slide 31 and/or the assembly cover 4 is made of a material or a combination of materials leading to good thermoconductivity properties, and/or an integrated heating and/or temperature measuring element, and/or at least one of the port 5 provided with a connecting means, such as a harness (tubes and fittings) suitable for automated application, and/or at least one port 5 that is provided with a removable closing 7 means such as a screw, and/or a sealing means 12, e.g. an O-ring, close to the port 5 suitable to provide a seal to closing or connecting means, and/or at least one fixing means 11 for fixing the microscope slide 3 on the assembly cover 4, such as O-rings.

The reaction chaser assembly 1 of the present invention can also comprise more than one reaction compartment 9, each comprising at least one channel 6, hereby the reaction compartment 9 can be separated or connected by one or more channels 6. The reaction compartment 9 may comprise an optionally removable shelf 13 to further reduce the reaction volume. In the case of a removable shelf 13, in one and the same assembly cover 4 different volumes can be adjusted.

The above described embodiments are described below in greater detail.

The assembly cover 4 is constructed in a manner that secures a pre-defined volume within a small-scale level and thus only requires minimal volumes of the reaction solution. As a consequence, costly biological material like antibodies, proteins or nucleic acids can be economized which is especially critical if the current invention is used for high through put applications.

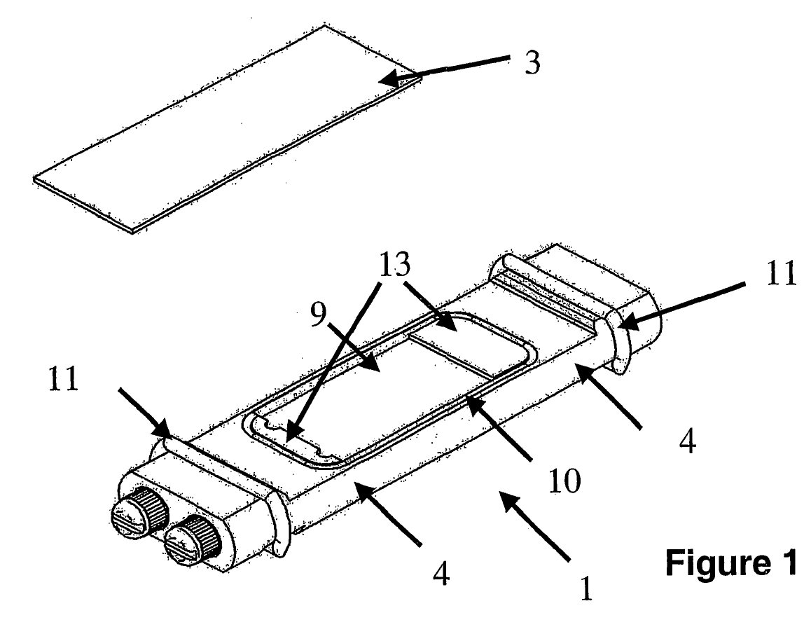

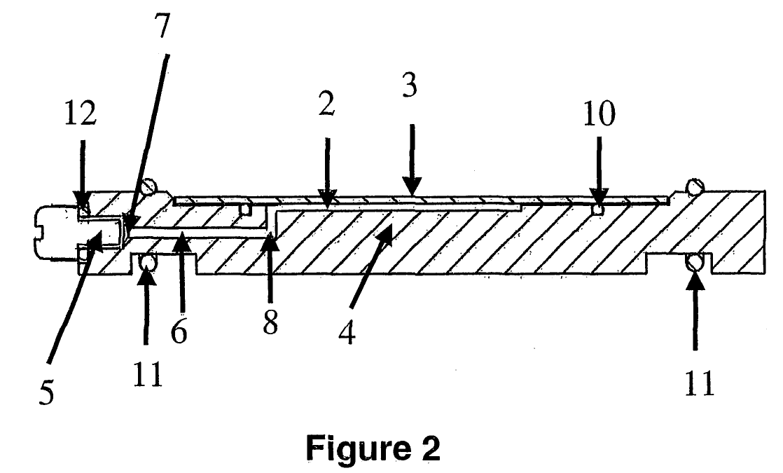

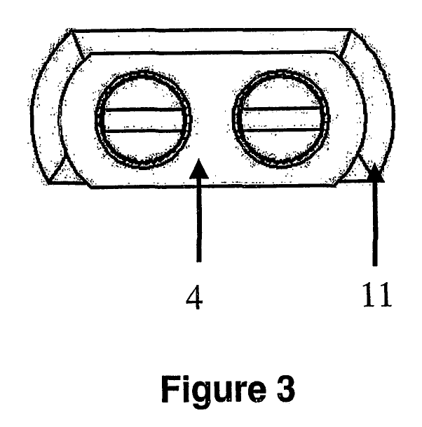

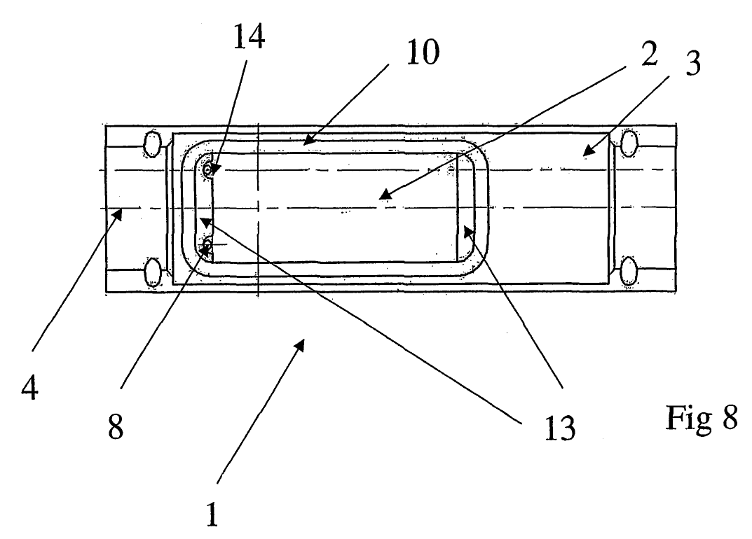

The assembly cover 4 of the present invention as e.g. shown in FIGS. 1 to 3, includes at least one port 5 and allows to load and unload the reaction chamber 2 without dismounting the system. To have a perfect seal the port 5 can be sealed by having an additional O-Ring 10. From each port 5 one small channel 6 leads to the reaction compartment 9 and in a preferred embodiment the channel 6 ends in a recess with a concave inlet 14 (second end 8 of channel 6) (FIG. 8). This is of importance to trap any air bubbles that are enclosed in the reaction chamber 2 and would interfere with the area that includes the biological specimen.

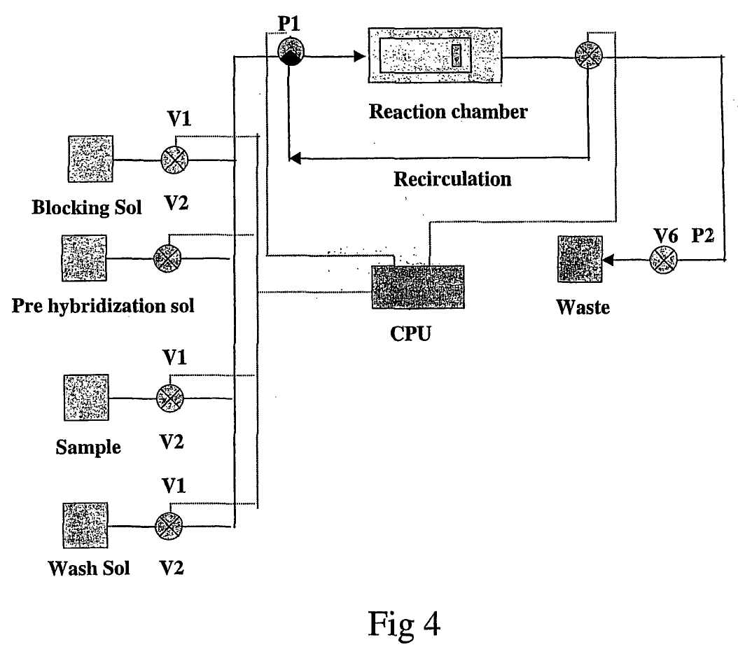

By having at least one, preferably at least two in/out ports, the system can be designed to run fully automated and controlled though an external device by pumping diverse solutions such as the reaction fluid or wash solutions in and out of the reaction chamber 2 (see FIG. 4). The ports preferably are positioned either at the same side or opposite to each other. A major advantage of having on and out ports is that the whole process of blocking, reaction, washing and detection performed during e.g. hybridization or antibody detection may be done in one single system without removing the slide which represents a major advantage over existing commercially available systems. While EP 1 160 612 A1 is suitable to perform hybridization reactions with microarray slides in a closed reaction volume, it has one major limitation. Loading and unloading of the sample needs to be done by puncturing the sealing casket which is not suitable for automatisation of the above mentioned variety of experimental procedures. While in U.S. Pat. No. 5,346,672 loading is performed through a concave opening and in U.S. Pat. No. 6,159,727 loading is done by opening the reaction chamber, no fluid flow through the reaction chamber is possible and thus it will not allow an automated procedure. In the current invention the reaction chamber can be run under a continuos fluid flow pathway, a major advantage for automated procedures.

A key feature of the present invention is mounting of the reaction containment system in an easy, safe and fast manner to secure a pre-defined closed reaction volume that prevents leakage of any sample material. This can be achieved by assembling the microscope slide 3 onto the O-ring 10 of the reaction chamber 2 and then fixing the microscope slide 3 to the assembly cover 4 with at least one further O-ring 11 that is rolled over the reaction chamber. Thus it is a far more easier system to handle compared to available or earlier described products or inventions.

Also encompassed by the present invention is an assembly cover 4 that is made of a thermoconductive material like e.g. polymethylmethacrylate (PMMA) and therefore allows optimal thermal contact with any heated surface through its flat shape at the bottom. Such design qualifies the reaction chamber 2 to be placed within the heating device of commercial thermocyclers like e.g. a Biometra “T-Gradient” if the heat control shall be performed by an external system. This further simplifies the process of hybridization by not having to use an oven with controlled temperature system. Furthermore the bioreactor can also be placed on any conventional rotisserie or water bath for hybridization.

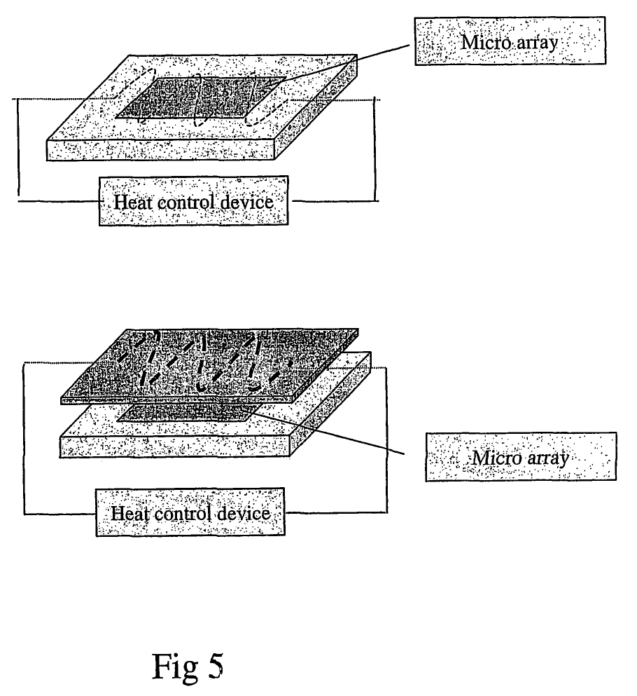

While other commercial available systems or the ones described in EP 1160 612 A1, U.S. Pat. No. 6,159,727, U.S. Pat. No. 5,346,672 allow hybridization to be performed, temperature control is only possible with external heating devices, the present invention also provides the possibile integration of an internal and infinitely variable thermoelement (FIG. 5 top) within the assembly cover 4. Another possibility to position the thermoelement is externally, namely on top of the microarray (see FIG. 5 bottom). This can be accomplished for instance by having a thermoconductive plate that can be attached additionally to the glass slide or positioned on the reaction assembly's slide bearing side after assembly, and controlled by an external device. The temperature of each chamber can be adjusted individually by an external control system that works like a computer or any other temperature control device

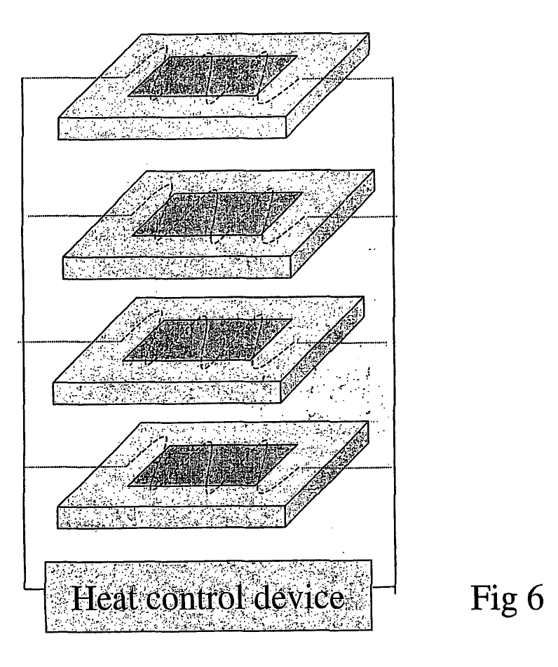

Another important aspect of the present invention is the design of the reaction chamber assembly 1 in a manner that it qualifies as a stand-alone product as described above but also has modular character. Thus it is possible to simply connect two or more of the reaction chamber 2 assemblies or bioreactor units in a way that they are placed side by side and/or head by head to form a 2D array (see FIG. 6). Through stacking the 2D array units it is even possible to form 3D arrays of the bioreactors.

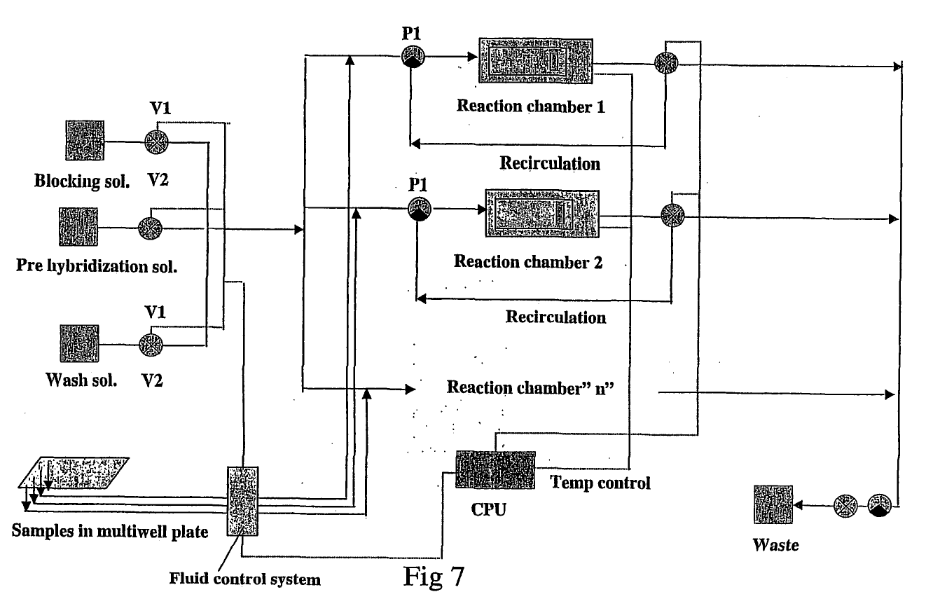

For an automated version of the reaction chamber 2, the in and out ports can be connected consecutively and fluid movement can be done by applying positive or negative pressure on the channels 6 (see FIG. 4). Any wash or incubation, reaction step can then be performed by an automated control device. This configuration allows to use the current invention also in high through put applications like e.g. drug screening, functional genomics and proteomics. Any of the described arrangements of bioreactor units can further be adapted to pipette robots or/and external heating devices (see FIG. 7). In the arrangement of FIG. 7, samples are prepared in e.g. multi-well plates, where each sample is loaded into a reaction chamber by an automated robotic system and processed according a defined protocol. While loading, blocking, pre-hybridization, hybridization, washing and temperature may be controlled by a engineered software, samples can either be processed serially or in parallel.

A further embodiment of the present invention provides a reaction chamber 2 inclusive optionally fixedly mounted harnesses (tubes, fittings, etc.) for automated applications. This may then be designed as disposable devices.

Two or more reaction chamber 2 assemblies can be parts of a modular system, whereby said assemblies should be preferably individually be controlled. In such modular system, the bioreactors can be placed in a housing of fixed or variable dimensions, said housing allowing easy connection and removal of the bioreactors and liquid supply units. Such housing optionally can provide an integrated heating and/or heat control system.

The reaction chambers, either in its stand-alone or in its more sophisticated modular version have several advantages over existing technologies.

While the systems described in EP 1 160 612 A1 and U.S. Pat. No. 6,159,727 allow reactions to be performed like e.g. hybridization of microarrays, they both require laborious intermediate steps such as dismounting the system in order to perform downstream processes like blocking, performing the biological reaction, washing and detection.

In e.g. EP 1160 612 A1 the sealing gasket needs to be punctured and systems that use a cover slip also do not qualify for an automated system. Furthermore, U.S. Pat. No. 6,159,727 is provided with a flange that is also not adequate for an automated system. The herein disclosed invention allows to perform all the above mentioned steps in a single all-in-one unit without the need to dismantle the system. This is a major advantage for setting up an automated version of the bioreactor systems, especially for high throughput applications.

A further advantage of the described invention is that biological reactions at elevated temperatures will not need to be performed in an additional moisture chamber. Other commercially available hybridization chambers for microarray applications are in principal moisture chambers, i.e. the microarray must be placed in a moisture chamber to prevent evaporation of the reaction fluid. In these systems the hybridization solution containing the labelled cDNA representing the target is incubated under a glass or plastic microscope slide 3 (coverslip) which is exposed to air and therefore the hybridization solution without specific provisions would evaporate quickly. In contrast, the reaction chambers of the invention enables to load minimal volumes of sample fluid and to keep it constant by preventing any evaporation of reaction fluid through the integrated sealing O-ring 10 on top of the cover assembly. Since handling with respect to loading the sample fluid is very easy through having an in and out port 5 that minimizes the risk of spillage significantly, it is also very suited for radioactive applications where spillage and safety aspects are of major importance. Systems that make use of cover slips or systems like those described in U.S. Pat. No. 6,159,727 are not adequate for radioactive procedures since handling becomes very difficult with radioactive labeled material.

Commercial applications of the devices of the current invention include in principal all biological reactions to be performed with any biological specimens mounted onto the surface of a rigid slide. A typical application is hybridization of nucleic acids e.g. in microarray applications where radioactive or fluorescence labeled cDNA is hybridized to an oligonucleotide probe printed on a glass microarray.

Beyond genomic applications the disclosed reaction chamber assembly 1 also can be used in the emerging field of proteomics where mostly protein protein interaction studies are performed to discover functional properties. One example is a recently discovered array technology for global protein expression analyses by BD Biosciences/Clontech. By using glass microscope slides 3 with hundreds of distinct antibodies bound to the surface of the slide, it has become possible to profile hundreds of native proteins simultaneously or to compare protein abundances in a variety of biological samples. Steps very well known for users skilled in the art like blocking, incubation, washing are usually performed in open incubation trays and are not economized with regard to manual handling, the amount of sample material and temperature control. The invention described here has overcome those limitations.

The current invention can be used in combination with various formats of cell-based assays taking place on a plastic/glass. microscope slide 3. Plastic or a special modified surface are well suitable to culture cells within a 2D environment and to study cellular phenomena. A recent publication by Ziauddin and Sabatini Nature 411:107; 2001 and U.S. 6,544,790 have shown that it is possible to print different CMV driven cDNA's plasmids on glass microarrays and in a second step to transfect cells directly on the array. Transfected cells that do express target molecules can then be detected by immunostaining applying conventional fluorescence microscopy. The described example is well suited to be performed within the device described in the current invention.

The current invention can also be used for screening purposes were e.g. glass microarrays are prepared with printed libraries of CMV driven cDNA's plasmids in combination with a key promoter-GFP plasmid. After finally assembly with cover 4 and adding transfection reagent, cells or a cell line may directly be seeded in the reaction chamber by an automated system. Following successful transfection positive interacting molecules from the expression library and the key promoter may then be screened under continuous fluid flow recirculation. Inducers of the corresponding promoter will lead to the expression of the reporter GFP molecule. Since the chosen material of the current cover assembly is transparent, positive cells can be detected directly through the reaction chamber by conventional fluorescence microscopy or CCD based detection systems without the need to disassemble the system. Such a process can also be performed by an automated fluorescence scanner allowing to measure GFP expression in real-time. By using a reaction chamber such as described in the current invention can drastically reduce consumption of expensive cell culture reagents, transfection reagents and other chemicals. Also less waste is produced if experiments are performed in smaller volumes. In addition a closed system such as the reaction chamber used in this experiment that can be viewed under a microscope without risk to contaminate the sample is much more convenient than a culture vessel based system.

A further application is to perform gene or protein expression analyses on tissue sections mounted onto glass microscope slides 3 in an easy-to use and temperature controlled manner. In situ hybridization and immunohistological experiments are representative examples.

Some of the mentioned applications, DNA and antibody microarrays and cellular assays are normally used for drug screening purposes where high throughput screening by means of a high degree of automatisation is a key issue The invention described herein provides a great improvement in this direction.

### EXAMPLES

### Abbreviations Used

SDS=sodium dodecyl sulfate

SSC=Saline-Sodium Citrate

BSA=bovine serum albumin

DMEM=Dulbecco's Modified Eagle Medium

EDTA=ethylenediamine tetraacetate

PCR=polymerase chain reaction

CMV=Cytomegalovirus

### Example 1

### Microarray Printing and Hybridization Process

Total RNA was isolated from human chondrocyte cells. RNA was reverse transcribed into cDNA using fluorescent Cy3 nucleotides to label the specific RNA probes. These probes were denatured and stored in hybridization solution containing 500 mM Sodium-Phosphate Buffer (pH 6.00, 1% SDS, 1% BSA, 1 mM EDTA).

Unlabeled 50mer oligonucleotides were spotted in 150 mM Sodium-phosphate buffer pH 8.5 at defined concentrations on commercially available epoxy coated standard microscope slides 3, permitted to dry in the humid chamber of the arrayer cabinet over night. The oligo arrays were then washed in 0.1×SSC, 0.1% SDS for two hours at room temperature and rinsed for 5 minutes in 0.1×SSC.

The microarray slides were then blocked in NoAb Blocking solution (NoAb Biodiscoveries). The reaction chamber 2 was washed with detergents, rinsed with Milli-Q water and rinsed again with 70% Ethanol to remove any remaining dust particle, fingerprints or similar. The screws were removed and the blocked microarray slide placed face down on the reaction chamber 2 of an assembly cover 4 as shown in FIGS. 1 to 3. The microarray slide was fixed with the clamping o-rings 11. The screws were removes to fill the chamber with hybridization solution. The hybridization solution was transferred with a 1000 ul micropipette and injected in one of the two channels 6, whereby the upper channel served as ventilation port 5. The reaction chamber 2 was kept in a 45° angle to let the air go out through the ventilation port 5 during hybridization solution injection. Another important step was to avoid air bubbles on the slide, because they may impair the outcome of the hybridization procedure. This was accomplished by keeping the chamber in 45-degree angle and by filling the reaction volume with hybridization solution up to the channels. By slightly pressing on the glass array trapped bubbles were then directed into the channels and subsequently the ports were closed. Additional small bubbles were then trapped in the concave recesses close to the channels.

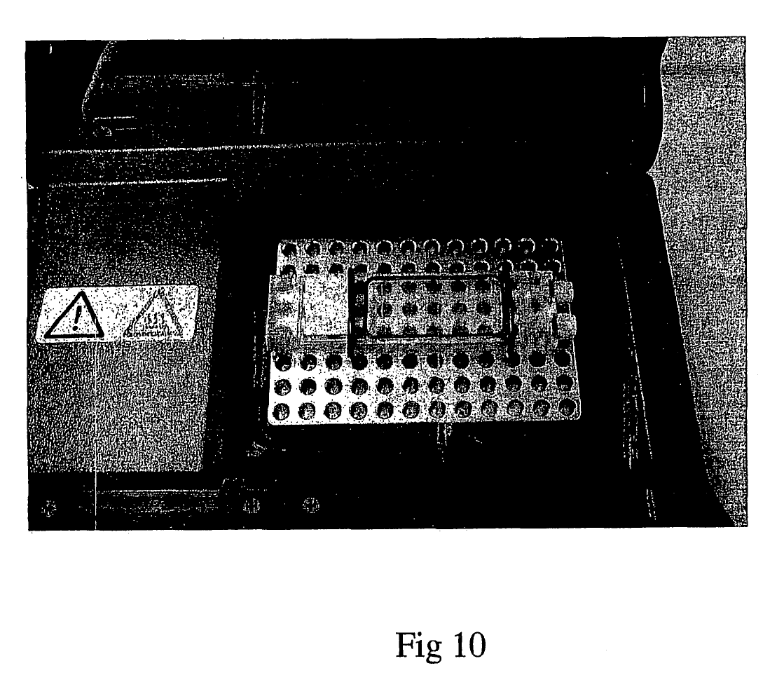

After sample loading the reaction chamber 2 was closed with both screws. The chamber was then ready for incubation at the appropriate temperature. For this step the reaction chamber 2 was placed in a standard 96well format PCR thermal cycler (TGradient, Whatman Biometra GmbH, Göttingen, Germany). Block and lid temperature were adjusted to 42° C. FIG. 10 shows a picture of a reaction chamber in a PCR thermal cycler prior to hybridization.

Incubation took place over night (12 hours).

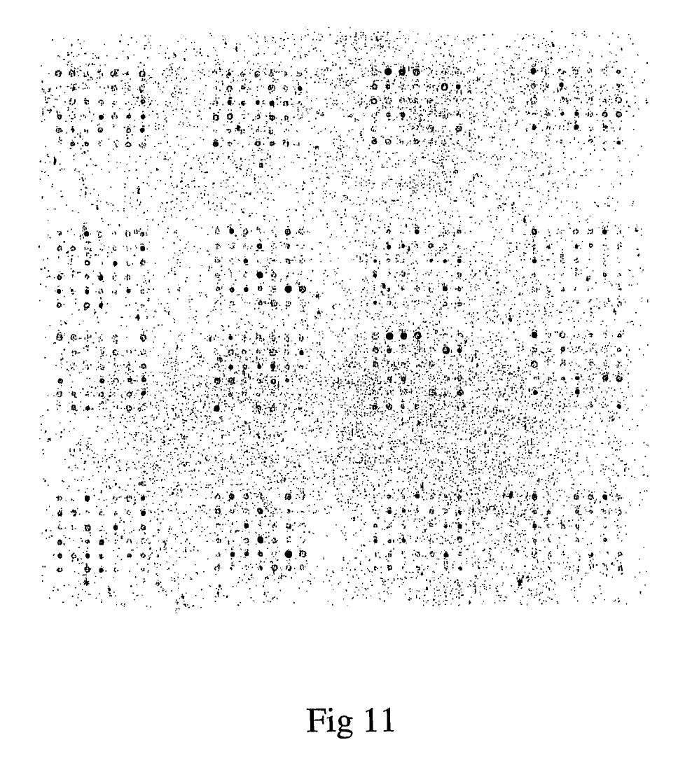

After this period the screws were removed and the hybridization solution containing the unbound Cy3-labeled cDNA was discarded. After incubation remaining unspecific probe was washed away with 1×SSC, 0.1%SDS for 1 hour at room temperature. The hybridized microarray was then scanned using Affymetriy 418 microarray. FIG. 11 shows a typcial result of such a scan. Every spot is representing one single gene. Different intensitites meaning different gene expression levels. For example a dark spot represents a high gene expression level where a weak spot represents low gene expression levels.

### Example 2

### Antibody Microarray

15 Million chondrocytes of two different samples were spun down in a microcentrifuge tube. Supernatant was completely discarded. After freezing the samples in liquid nitrogen cell pellets were placed at room temperature. 20 μl of Extraction Buffer provided with BD Clontech™ Protein Extraction & Labeling Kit was added per mg of cells. Lysate was thoroughly mixed by vortexing. The homogenous samples were then incubated at room temperature for 10 min with slow rotation. Lysate was centrifugated for 30 min at 10,000×g at 4° C. Supernatant was carefully removed and transferred to another clean tube. Protein concentration was measured using standard Bradford assay. Sample was diluted with Extraction Buffer to 1.1 mg/mL.

Each vial of Cy3 mono reactive dye and Cy5 mono reactive dye (Amersham Pharmacia Biotech) was dissolved in 50 μl Labeling Buffer. Cy3 dissolved in 50 μl Labeling buffer was immediately added to 1 mg protein of one sample and Cy5 dissolved in 50 μl Labeling buffer was added to the other sample, both extracted with BD Clontech™ Protein Extraction & Labeling Kit (see above). The samples were mixed by inverting the tube 3 times. Drops were collected at the bottom of the tube by short centrifugation. Labeling reaction took place at 4° C. for 90 min. Tube was mixed by inversion every 20 min to improve dye coupling.

4 μl of Blocking Buffer was added to each sample, mixed by inverting the tubes and incubated for 30 min. During incubation the tubes were inverted every 10 min to improve blocking.

Millipore Microcon Concentrators columns were used to remove unbound dye molecules for each sample. Cocentrate was diluted in 20 μl 1× Desalting Buffer, centrifuged back to a fresh microtube and both samples pooled together.

One Antibody Microarray was placed upside-down on sealing o-ring of an assembled reaction chamber and fixed with two clamping o-rings. Antibody Microarray was blocked by injection of 900 μl Blocking Buffer into one injection port. The other port served as venting channel. Both ports were closed with provided screws The Microarray was blocked for 30 min at room temperature.

Blocking Buffer was replaced with 900 μl Incubation Mix containing 10 μg differentially labeled and desalted protein prepared above. Incubation Mix was incubated for 30 min at room temperature, replaced with Wash Buffer and incubated for 15 min at room temperature. This step was repeated two more times.

After incubation and washing the Antibody Microarray was removed and centrifuged at 1000×g for 25 min at room temperature to remove remaining water droplets.

The dried array was scanned within 24 hours in a Genetic Micro Systems (GMS) scanner at 10 μm resolution to obtain a two color image consisting of one channel for Cy3 sample and another channel for Cy5.

The so labeled protein samples were easily hybridized in this kind of reaction chamber. The small volume provided by the reaction chamber leads to uniform and highly reproducible, differentially labeled antibody microarrays compared to alternative methods such as cover slip incubation.

### Example 3

### Detection of Collagen Type 2 in Human Cartilage Tissue Sample Extracts

Total protein was extracted from different human cartilage tissues. Samples were transfered onto NoAb Epoxy Activated Slide UAS0005E (Noab Biodiscoveries, Mississauga, Ontario, Canada) according to protocol.

The slide was placed upside down in a reaction chamber and fixed with clamping o-rings. To prevent unspecific antibody coupling the membrane was blocked in 900 μl TBS containing 2% non-fat milk powder for 2 hours at room temperature.

Primary antibody mix was obtained by diluting 1 μl specific collagen II Ab-2 antibody (Novocastra Laboratories Ltd., Newcastle upon Tyne U.K.) in 900 μl TBS containing 0.5% non-fat milk powder. Blocking solution was removed from reaction chamber, replaced with primary antibody mix and incubated for 2 h at room temperature. Primary antibody mix was removed, the reaction chamber filled with TBS and incubated for 2 min. This step was repeated four times.

Secondary antibody mix was obtained by diluting 1 μl secondary antibody coupled to alkaline phosphatase enzyme in 900 μl TBS containing 0.5% non-fat milk powder. TBS was removed from reaction chamber, replaced with secondary antibody mix and incubated for another 2 h at room temperature. Secondary antibody mix was removed and reaction chamber rinsed with TBS for 2 min. This step was repeated four times to completely remove all remaining antibodies.

After coupling of primary and secondary antibody the slide was developed. 5 μl NTB (nitroblue tetrazolium chloride) and 3.4 μl BCIP (5-bromo, 4-chloro, 3-indolylphosphate) were diluted in AP Substrate Buffer (100 mM TRIS®, 100 mM sodium chloride, pH 9.50) and injected into reaction chamber for development. After developing the slide was removed from reaction chamber and briefly washed with TBS and dried at room temperature.

A picture of the membrane slide was taken that was analyzed in a densitometry software. The more blue color from developed nitroblue tetrazolium chloride the more collagen type 2 was present in a single spot representing a specific tissue sample.

Significant reduction of reagents (especially very expensive ones such antibodies or enzymes) was achieved by using such a reaction chamber.

### Example 4

### In situ Hybridization of Digoxigenin-UTP (DIG) Labaled Collagen Type 2 RNA In Chondorcyte Pellet Culture

A collagen type 2 (Col-II) cDNA clone fragment was subcloned into a polylinker site of a pSPT18 transcription vector which contains a promotor for T7 and SP6 RNA polymerases. After linearization of template DNA an RNA polymerase was used to produce transcripts. DIG-UTP served as a substrate and was incorporated into the transcript.

Template DNA was linearized with Eco RI restriction enzyme and purified. 1 g of linearized and purified template DNA was diluted in 13 μl nuclease free water. 2 μl 10×NTP Labeling Mixture, 2 μl 10× Transcription Buffer and 1 μl of RNase Inhibitor was added to the template and mixed gently. 2 μl T7 RNA Polymerase was added. The reaction was gently mixed, spun down to collect droplets at the bottom of the tube and incubated for 2 hours at 37° C. After amplification labeled template DNA was digested using 2 μl DNase I for 15 min at 37° C.

The reaction was stopped by adding 2 μl 0.2M EDTA (pH 8.00).

Paraffin embedded tissue sections of chondrocyte pellet cultures on silane-coated microscope slides were used for detection of Col-II in these samples. Sections were incubated in PBS Buffer (140 mM sodium chloride, 2.7 mM potassium chloride, 10 mM di-sodium hydrogen phosphate, 1.8 mM Potassium-dihydrogenphosphate at pH 7.40) two times for 5 min and in PBS containing 100 MM glycine other two times for 5 min. After this first incubation sections were treated with PBS containing 0.3% Triton X-100 and washed for two times 15 min in PBS. Sections were permeabilized for 30 min at 37° C. in TE Buffer [100 mM TRIS®, 50 mM Ethylendiamine-tetra-acetic acid (EDTA), pH 8.00] containing 10 μg/mL RNase-free Proteina-se K Sections were then post-fixed for 5 min at 4° C. in PBS containing 4% paraformaldehyde. Sections were washed two times for 5 min in PBS and acetylated in TAE Buffer [100 mM, Tri-ethanolamine, pH 8.00 containing 0.25% (v/v) acetic anhydride] two times for 5 min.

This slide supporting a post-fixed tissue section was placed upside-down in a reaction chamber and fixed with clamping o-rings. Prehybridization buffer [4× saline sodium citrate (1×SSC=150 mM sodium chloride, 15 mM sodium citrate at pH 7.20) containing 50% (w/v) deionized formamide] was injected through one of both ports. The ports were closed using screws and the slide was incubated at 37° C. for 15 min.

Prehybridization Buffer was replaced by Hybridization Buffer [containing 40%. deionized formamide, 10% dextran sulfate, 1× Denhardt's solution, 4×SSC, 10 mM Dithio-threitol, 1 mg/ml yeast t-RNA and 1 mg/ml denatured and sheared salmon sperm DNA] including amplified and labeled template RNA and incubated overnight at 42° C. in a closed and evaporation protected environment.

For posthybridization the reaction chamber was rinsed with 2×SSC (see above). The reaction chamber was emptied filled again with 2×SSC and incubated at 37° C. in 2×SSC in a hybridization oven for 30 in. This step was repeated with 2×SSC and then repeated with 1×SSC for two times. To digest any single-stranded (unbound) RNA probe, sections were incubated for 30 min in NTE Buffer (500 mM sodium chloride, 10 mM Tris, 1 mM EDTA, pH 8.00) containing 20 μg/mL RNase A. After digestion the slide was washed two times in 0.1×SSC for 30 min at 37° C. in a shaking waterbath.

For immunological detection slides were washed in Buffer 1 (100 mm TRIS® pH 7.5, 150 mM sodium chloride) two times at room temperature for 10 min. Sections were covered for 30 min at room temperature with Blocking Solution (Buffer 1 containing 0.1% Trition X-100 and 2% sheep serum). Decant Blocking Solution and incubate slides in a reaction chamber with buffer 1 containing 0.1% Triton X-100, 1% normal sheep serum, and a suitable dilution of sheep anti-DIG-alkaline phosphatase antibody (diluted 1:1000). A rocking platform was used to wash sections two times in Buffer 1 for 10 min. Buffer 1 was discarded and sections were incubated for 10 min in Buffer 2 (100 mM TRIS® pH 9.50, 100 mM sodium chloride and 50 mM magnesium chloride). The chamber was completely drained and immediately filled with 900 μl Staining Solution [890 μl Buffer 2 (see above), 4 μl nitroblue tetrazolium (NBT, 75 mg/mL in 70% dimethylformamide), 3.15 μl 5-bromo-4-chloro-3-indolyl-phosphate (BCIP or X-phosphate, 50 mg/mL in 100% dimethylformamide) and 1 mM levamisole]. Reaction chamber was closed and incubated for approximately 6 h in a dark place until development was complete.

Color development was stopped by replacing Staining Solution with Buffer 3 (10 mM TRIS® pH 8.10, 1 mM EDTA). The slide was then removed from reaction chamber and dipped briefly in distilled water.

The images can then be viewed under a fluorescence microscope.

### Example 5

### Semi-Automated System With Temperature Control

A reaction chamber was modified as follows: A) A negative temperature coefficient thermistor (NTC type B57861-S103-F40, Epcos, Munich Germany) was added for inside chamber temperature control and connected to a multimeter for resistance measurement. B) Both screws which close the port channels were replaced by screws modified to be able to connect tubings. One of said ports was defined as inlet port. Hybridization and wash solutions were injected by using a peristaltic pump through this inlet port. The other tube was defined as waste port used for hybridization and wash solution outlet through a connected tubing.

NTC thermistor resistance was calibrated between 25° C. and 50° C. in a reference system using a standard multimeter (Metex M-4650CR, Metex, Seoul, Korea).

The reaction chamber was placed in a standard PCR thermal cycler (TGradient, Whatman Biometra GmbH, Göttingen, Germany) to keep temperature stable at 42° C./50° C. equal to 5.05/3.63 kOhm measured with said multimeter connected to the NTC thermistor.

The final semi-automated system consisted of a reaction chamber connected to a tubing system for hybridization or wash solution inlet and outlet, an NTC thermistor connected to a multimeter-for temperature measurement and a thermal cycler to provide a precise temperature environment of 42° C./50° C. (which is a commonly used temperature setting in experiments described in examples 1, 2 and 3).

Hybridization solution was injected into the assembled system through the inlet port by turning on the peristaltic pump until the reaction chamber was completely filled with hybridization solution. A previously set temperature was generated by the used PCR thermal cycler and set temperature was in turn controlled by an independent system.

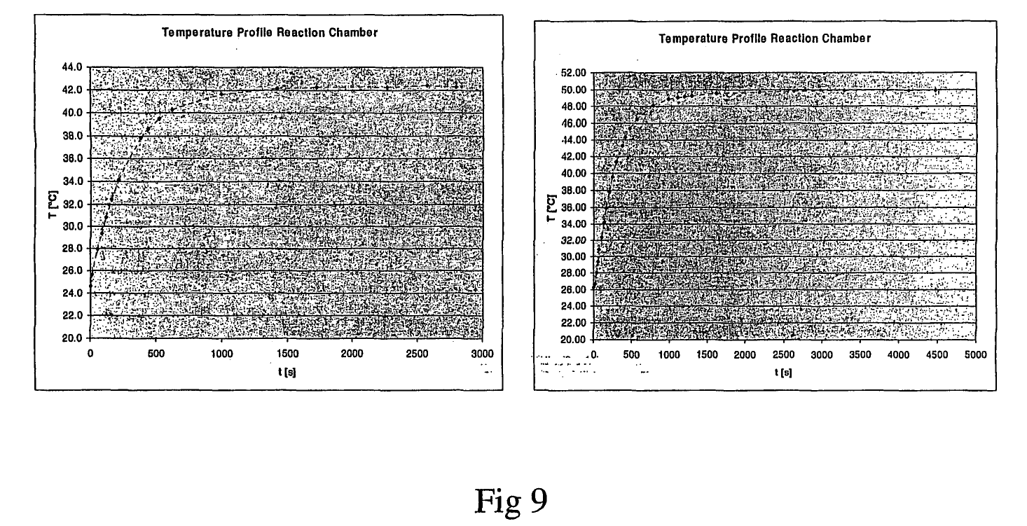

The first temperature profile (see FIG. 9, left side) shows a reaction chamber specific temperature curve over time. After 15 to 20 min the set temperature of 42° C. was reached inside the chamber depending on chamber content and environmental temperature.

The second temperature profile (see FIG. 9, right side) shows a temperature profile for another set temperature of 50° C.

The current examples show the implementation of the reaction chamber into a system that allows to perform a controlled process in an automated manner.

### Example 6

### Cell Based Microarray

This example shows the use of the described reaction chamber in combination with cell based reporter assays. Cells can be viewed conveniently inside a closed reaction chamber under aseptic conditions.

A Collagen-1 promotor was subcloned into a mammalian vector expressing GFP as fluorescent molecule. The COLLAGEN-1 promoter-reporter construct was diluted in 0.2% gelatin at a concentration of 40 ng/μl. This spotting solution was spotted with a micropipet tip onto a glass microscope slide Spotted slides were dried, viewed under a light microscope for quality control and stored for further use at 4° C. Chondrocyte cells were proliferated in DMEM (containing 10% Fetal Calf Serum and antibiotics) until 80% confluence. Cells were detached and spun down in a centrifuge for 10 min at 300×g. Medium was replaced by fresh culture medium and cells stored for injection into chamber

One microarray was placed upside-down on a sealing o-ring of an assembled reaction chamber and fixed with two provided clamping o-rings. 300 μl cell culture medium containing 2 μl FuGene 6 Transfection Reagent (Roche, Basel Switzerland) was injected into a reaction chamber and incubated for 15 min at roam temperature. After incubation 600 μl cell culture medium as described above containing 1 million chondrocytes was injected into the reaction chamber and incubated at 5% carbon dioxide at 37° C. After cell attachment (at least overnight incubation) the microarray slide was washed by careful injection of 5 ml Phosphate Buffered Saline (PBS) . After rinsing the reaction chamber the whole chamber was turned upside down to view the cell microarray under a reverse fluorescence microscope.

1. A reaction chamber assembly comprising e.g. a microscope slide or any other slide or carrier system and an assembly cover, wherein said assembly cover comprises at least one port and at least one channel having a first end at the port and a second end at a reaction compartment which reaction compartment together with the microscope slide forms a reaction chamber with predetermined volume.

2. The reaction chamber assembly of claim 1, wherein said channels at their second end are formed such that they end in a recess with a concave inlet.

3. The reaction chamber assembly of the preceding claims, wherein the assembly cover comprises an O-ring surrounding the reaction compartment and establishing a seal to the microscope slide.

4. The reaction chamber assembly of anyone of the preceding claims, wherein the assembly cover is made of a material or a combination of materials leading to an assembly cover with good thermoconductivity, e.g. polymethylmethacrylate.

5. The reaction chamber assembly of anyone of the preceding claims, wherein the assembly cover is made of a material or a combination of materials leading to an assembly cover with optimal transparent properties in order to be used within optical detection systems e.g. fluorescence microscopes or CCD based detection systems.

6. The reaction chamber assembly of anyone of the preceding claims, wherein at least one of the port(s) is provided with a connecting means, such as a harness suitable for automated application.

7. The reaction chamber assembly of anyone of the preceding claims, wherein a heating element and/or temperature measuring element is integrated in the assembly cover.

8. The reaction chamber assembly of anyone of the preceding claims, wherein at least one of the ports is provided with a removable closing means such as a screw.

9. The reaction chamber assembly of anyone of the preceding claims, wherein a sealing means, e.g. an O-ring, is provided close to the port suitable to provide a seal to closing or connecting means.

10. The reaction chamber assembly of anyone of the preceding claims, wherein said assembly cover is provided with at least one clamping means for fixing the microscope slide on the assembly cover, such as O-rings.

11. The reaction chamber assembly of anyone of the preceding claims, wherein the assembly cover comprises at Least two ports and at least two channels ending in the same reaction compartment.

12. The reaction chamber assembly of anyone of the preceding claims, wherein the assembly cover comprises at least two reaction compartments.

13. The reaction chamber assembly of anyone of the preceding claims, wherein the reaction compartment comprises a shelf.

14. The reaction chamber assembly of claim 13, wherein the shelf is variable.

15. The reaction chamber assembly of anyone of the preceding claims, wherein the microscope slide carries biological specimen.

16. A modular system comprising at least two reaction chamber assemblies of anyone of the preceding claims, wherein any one of the bioreactors can individually be removed.

17. The modular system of claim 16, wherein the bioreactors are placed in a housing of fixed or variable dimensions, said housing allowing easy connection and removal of the bioreactors and liquid supply units.

18. The modular system of claim 16 or 17, wherein said housing provides a temperature adjusting system.

19. An assembly cover comprising at least one port and at least one channel having a first end at the port and a second end at a reaction compartment which reaction compartment together with the microscope slide forms a reaction chamber with predetermined volume.

20. The assembly cover as defined in anyone of claims 2 to 15.

21. A temperature controlling and adjusting cover with an at least in part planar surface that can be brought in contact with the microscope slide or the assembly cover of the bioreactor of anyone of claims 1 to 15 in at least the region of the reaction chamber.

22. A temperature controlling and adjusting system comprising at least two temperature controlling and adjusting covers of claim 21.
